(19) United States
(12) Patent Application Publication
(10) Pub. No.: US 2024/0122485 A1
(43) Pub. Date: Apr. 18, 2024

(54) PHYSIOLOGICAL ANALYSIS USING WEARABLE SENSOR ARRAY

(71) Applicant: Human Systems Integration, Inc., Walpole, MA (US)

(72) Inventors: Brian Farrell, Walpole, MA (US); Matt Abelson, Walpole, MA (US); Viprali Bhatkar, Walpole, MA (US); Allan Neville, Walpole, MA (US); Richard Gerard Streeter, Walpole, MA (US); Karin Radrich, Walpole, MA (US); John Phillips, Walpole, MA (US)

(21) Appl. No.: 17/768,697

(22) PCT Filed: Oct. 13, 2020
(86) PCT No.: PCT/US20/55390

§ 371 Date: Apr. 13, 2022

Related U.S. Application Data
(60) Provisional application No. 62/914,507, filed on Oct. 13, 2019.

(51) Int. Cl. A61B 5/0205; A61B 5/00; A61B 5/11

(52) U.S. Cl. CPC A61B 5/0205; A61B 5/1116; A61B 5/1118; A61B 5/1123; A61B 5/6804; A61B 5/7221; A61B 5/7282; A61B 5/7405; A61B 5/742; A61B 5/7455; A61B 5/02405; A61B 2560/0214; A61B 2560/0443; A61B 2562/063; A61B 2562/066; A61B 2562/164; A61B 2562/227

(57) ABSTRACT

Disclosed embodiments describe techniques for physiological analysis. The physiological analysis is based on the use of a wearable sensor array. A plurality of sensors and conductors is coupled to a compression garment, where the compression garment has stretchable portions that stretch, e.g., in a single dimension. The garment can include a shirt, a sports bra, or a vest. Associated on-board electronics are mounted to at least one of the compression garment and the hub and electrically connected to the hub and generate a physiological profile, based on at least one output from each of the sensors. At least one aspect of the physiological profile is communicated to a processor configured to analyze the at least one aspect and generate an assessment based on the analysis.

### REFERENCE TO PENDING PRIOR PATENT APPLICATION

This patent application claims benefit of pending prior U.S. Provisional Patent Application Ser. No. 62/914,507, filed Oct. 13, 2019 by Human Systems Integration, Inc. and Brian Farrell et al. for PHYSIOLOGICAL ANALYSIS USING WEARABLE SENSOR ARRAY (Attorney's Docket No. HSI-002Q), which patent application is hereby incorporated herein by reference.

### FIELD OF ART

This application relates generally to physiological analysis and more particularly to physiological analysis using wearable sensor arrays.

### BACKGROUND

People around the world live, work and play in a wide range of environments. The environments can range from the hot, arid conditions of a desert, to the hot, humid conditions of a jungle, to the frosty, low-humidity conditions of high mountains or polar regions. Whatever the environment, proper clothing is demanded for comfort, safety or even survival. The clothing that people choose to wear in their environments is often dictated by culture, local requirements, or fashion. Some cultures prescribe dress codes for what is considered proper attire for women and men, while other cultures maintain a laissez faire attitude. Similarly, light-weight, loose-fitting clothing is most comfortable in the tropics, in contrast to the heavy wool or fleece sweaters and jackets worn in cold climes. And in terms of fashion, wearing a couture gown and extravagant jewelry, or wearing a black tie and a diamond-accented dress watch, may be highly appropriate for a red carpet or a gala affair, although such attire would be ludicrous or even dangerous in the Antarctic. People thus choose and wear their clothing to meet these many diverse requirements. In many cases, the clothing choices come down to personal preference, clothing price point, or even individual sense of fun. An otherwise drab or muted outfit can be enlivened by a colorful scarf, a brightly patterned shirt, or a delightfully loud tie.

### SUMMARY

Physiological analysis is based on using a wearable sensor array. An array of sensors can be coupled to a garment such as a compression garment. The sensors can be connected to a hub (or hubs) on the garment using conductors, where the conductors can include serpentine conductors, curved serpentine conductors, and so on. The sensors can include one or more of skin temperature sensors, galvanic skin response sensors, blood volume sensors, activity sensors, heart rate sensors, respiration sensors, and the like. The serpentine conductors can stretch or move in compliance with the stretch garment without breaking or losing conductivity. The serpentine conductors can form, in a macro sense, a variety of shapes or geometries including a straight line, a horseshoe, a sine or zigzag pattern, and so on. The conductors can be coupled to a stretch fabric that forms the compression garment or other garment. The conductors can be coupled to the garment using techniques that can include printing the conductors onto the fabric, pressing or attaching the conductors to the fabric, weaving the conductors into the fabric, etc. Thus, the wearable sensor array can comprise a plurality of sensors, a plurality of conductors and a hub. Associated on-board electronics can be mounted to the garment. These associated on-board electronics enable a physiological profile to be generated, based on at least one output from the sensors. In one form of the invention, these associated on-board electronics are mounted to the garment by forming the associated on-board electronics as part of the sensor array, e.g., as part of the hub. In another form of the invention, these associated on-board electronics are formed separately from the sensor array and are physically mounted to the garment and electrically connected to the sensor array (e.g., by electrically connecting the associated on-board electronics to the hub). In one preferred form of the invention, these associated on-board electronics are in the form of a physiological unit computer (PUC) that is physically and mechanically releasably mounted to the hub. Aspects of the physiological profile generated by the associated on-board electronics can be sent to a processor such as a computing device (e.g., a computer), a personal electronic device (e.g., a smartphone or tablet), etc., for analysis and generating of a physiological assessment. If desired, actuators can also be attached to the garment and actuated via the hub. By way of example but not limitation, a haptic device may be included as part of the wearable sensor array and used to provide feedback to the individual.

Thus it will be seen that the associated on-board electronics are interposed between the sensors and the processor. The sensors provide anatomical data to the associated on-board electronics, and the associated on-board electronics use this data to provide a physiological profile for the wearer of the garment. The associated on-board electronics pass the physiological profile to the processor, which uses the physiological profile to generate a physiological assessment of the wearer of the garment. The associated on-board electronics are mounted, either permanently or temporarily, to the garment (e.g., in one form of the invention, the associated on-board electronics are contained in the PUC, which is removably connectable to the hub, which is itself connected to the garment); and the processor is located “off garment”, with the associated on-board electronics passing the physiological profile to the processor using wired or wireless schemes so that the processor can generate a physiological assessment of the wearer.

In embodiments, a system for physiological analysis comprises: a plurality of sensors and conductors attached to a compression garment, wherein the compression garment has stretchable portions that stretch in one or more dimensions, wherein the conductors are connected to a hub; and associated on-board electronics (e.g., the PUC) configured to be permanently or temporarily attached to the conductors (e.g., at the hub) and generate a physiological profile of the wearer based on at least one output from each of the sensors. The physiological profile is transmitted from the associated on-board electronics (e.g., the PUC) to a processor for generating a physiological assessment of the wearer.

In embodiments, an apparatus for physiological analyses comprises: a plurality of sensors coupled to a garment, wherein the sensors include: a heart rate sensor; a respiration sensor; a body temperature sensor; and an orientation sensor; conductors for connecting the plurality of sensors to a hub; and associated on-board electronics designed and configured to be permanently or temporarily attached to the conductors (e.g., at a hub) and to generate a physiological profile of the wearer based on at least one output from each of the sensors.

In one form of the invention, there is provided a method for physiological analysis, the method comprising:
mounting a plurality of sensors, a plurality of conductors, and a hub to a compression garment, wherein the plurality of conductors electrically connect the plurality of sensors to the hub, and mounting associated on-board electronics to at least one of the compression garment and the hub and electrically connecting the associated on-board electronics to the hub;
using at least one sensor to acquire physiological data from a wearer of the garment;
using the associated on-board electronics to generate a physiological profile based on at least one output from the plurality of sensors;
communicating at least one aspect of the physiological profile to a processor located off garment; and
using the processor to analyze the at least one aspect of the physiological profile and generate a physiological assessment based on the analysis.

In another form of the invention, there is provided a system for physiological analysis comprising:
a compression garment;
a plurality of sensors mounted to the compression garment;
a plurality of conductors mounted to the compression garment;
a hub mounted to the compression garment, wherein the plurality of conductors electrically connect the plurality of sensors to the hub;
associated on-board electronics mounted to at least one of the compression garment and the hub, wherein the associated on-board electronics are electrically connected to the hub; and
a processor located off garment;
wherein at least one of the plurality of sensors acquires physiological data from a wearer of the compression garment;
wherein the associated on-board electronics generate a physiological profile based on at least one output from the plurality of sensors; and
wherein the processor analyzes the at least one aspect of the physiological profile and generates a physiological assessment based on the analysis.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for physiological analysis using a wearable sensor array.

FIG. 2 illustrates a state diagram of the PUC electronics.

FIG. 3 shows electrodes, serpentine conductors (in both straight and curved configurations), and a hub.

FIG. 4 illustrates a unisex garment and sensor array, e.g., sensors, conductors, and a hub.

FIG. 5 shows a female garment and sensor array, e.g., sensors, conductors, and a hub.

FIG. 6 shows a PUC and charging dock.

FIG. 7 is a block diagram showing data processing in the system.

FIG. 8 is a system diagram for physiological analysis using a wearable sensor array.

### DETAILED DESCRIPTION

Whether working, recreating or on maneuvers in any environment, proper clothing is required to remain cool or warm, to provide comfort, to perform a task, or for protection. In fact, proper clothing is so critical to such endeavors that the clothing can easily mean the difference between life and death. The deleterious effects of extreme temperatures on a human body are well known. Principal among these effects are frostbite and hypothermia from cold, and burns from heat. Frostbite can cause damage to exposed tissue, and can easily occur in digits, ears, nose, or any exposed skin. Damaged tissue at a digit due to cold or heat can cause difficulty in moving a digit, and in extreme cases, the loss of the digit or appendage. Hypothermia can result from the body temperature of a person dropping below 95 degrees Fahrenheit (35 degrees Celsius). Further risks of cold or heat can include heat stroke or problems with the heart. The extreme temperatures can cause the heart to pump harder as the body attempts to maintain core temperature or to remove heat from the core. The commensurate increases in heart rate and blood pressure can cause a heart attack. Clearly, physiological monitoring of an individual is critical in an environment that can kill.

Civilians, athletes, emergency services personnel, first responders, law enforcement personnel, and military personnel must wear clothing that is appropriate for the tasks the people are performing. Many activities can be potentially hazardous such as exercising heavily in high heat, at altitude, and so on. Other activities can be extremely hazardous, such as firefighting, riot control, or combat. The health and welfare of an individual performing risky tasks or operating in high risk environments are paramount. In order to track the health and welfare of individuals, sensors, conductors and hubs can be applied to the clothing worn by the individuals. As hereinafter disclosed, physiological analysis may be effected using a wearable sensor array (which comprises the sensors, conductors and a hub). The sensors are mounted to a garment and can monitor body temperature, determine respiratory rates and heart rates, and so on. The sensors can further monitor orientation or motion to detect a “soldier down” scenario. Conductors mounted to the garment transmit data from the sensors to a hub also mounted to the garment. Data can be collected from the hub using associated on-board electronics designed to be permanently or temporarily attached to the sensor array (e.g., incorporated in or attached to the hub), and hence attached to the garment. The sensor data can be analyzed by the associated on-board electronics to determine a profile such as a physiological profile for the individual. Aspects of the physiological profile, such as temperature, heart rate, or respiratory rate, can be analyzed (e.g., by a processor), and a physiological assessment can be generated. If desired, actuators can also be attached to the garment and actuated via the hub. By way of example but not limitation, a haptic device may be included as part of the wearable sensor array and used to provide feedback to the individual.

Thus it will be seen that the associated on-board electronics are interposed between the sensors and the processor. The sensors provide physiological data to the associated on-board electronics, and the associated on-board electronics use this data to provide a physiological profile for the wearer of the garment. The associated on-board electronics pass the physiological profile to the processor, which uses the physiological profile to generate a physiological assessment of the wearer of the garment. The associated on-board electronics are mounted, either permanently or temporarily, to the garment (e.g., in one form of the invention, the associated on-board electronics are contained in the PUC, which is removably connectable to the hub, which is itself connected to the garment), and the processor is located “off garment”, with the associated on-board electronics passing the physiological profile to the processor using wired or wireless schemes.

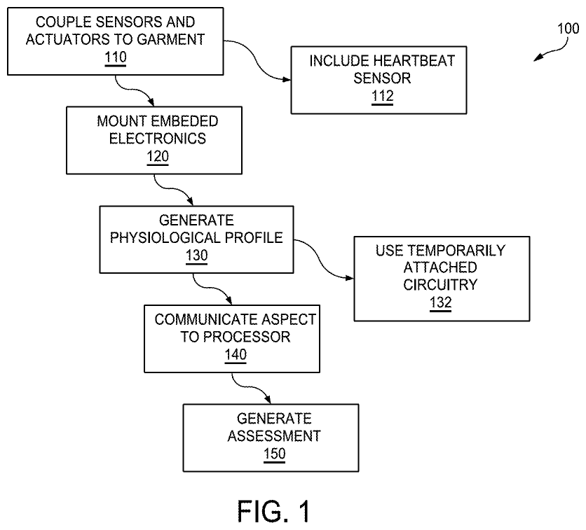

FIG. 1 is a flow diagram for physiological analysis using a wearable sensor array. The wearable sensor array can be attached to a garment such as a compression garment. People wear a variety of types, sizes, shapes, and colors of garments or other wearable items such as shoes or boots, hats or balaclavas, helmets, and so on. The particular garments or wearable items that a person wears may be dictated by local custom, may be chosen to communicate or convey messages to other people, or may be required based on climate, activity, profession, and so on. In hot climates, the garments and other wearable items that a person wears can be chosen to shield the body from direct sun, wick moisture away from the skin to aid cooling, and so on. In cold or even dangerous climates, the garments and other wearable items can be chosen to provide protection against hostile environmental elements. The items can include hats, gloves, gaiters, windproof garments, boots, etc. Whether to protect against heat or cold, garments are donned to keep wind and precipitation away from the body, while at the same time stabilizing body heat and dry comfort. Anyone familiar with outdoor activities is aware that a person who becomes overheated and damp from exercising runs a significant risk of exposure, hypothermia, or frostbite when the activity abates. To allay this problem, specialty fabrics have been developed that purport to keep exterior moisture out while wicking internal moisture away from the body.

Sensing and analyzing physiological aspects of a person performing a variety of tasks pose significant design and implementation challenges. The garments that are worn can be highly specialized for tasks or activities that the person performs. Further, the activities can include frequent movement, wide ranges of motion, and other motion that can easily cause connections or attachments to sensors to fail. Careful decisions must be made with respect to the “right” choices of connections, conductors, and fabrics. In addition, the sensors, attachments, conductors, etc., must not limit or restrict critical tasks or activities undertaken by the person. The critical tasks can range from fire suppression on an oil rig in the North Sea or in the Arctic, to missions of military personnel in cold or wet environments, to law enforcement activities, and the like. The present invention discloses approaches which enable a garment (such as a compression garment) to remain lightweight, warm, stretchable, etc., while carrying a wearable sensor array. A plurality of sensors and conductors are coupled to a compression garment, wherein the compression garment has stretchable portions that stretch (e.g., in a single dimension), and wherein the conductors are connected to a hub. Associated on-board electronics are permanently or temporarily connected to the sensor array (e.g., incorporated in or attached to the hub). These associated on-board electronics enable a physiological profile to be generated, based on at least one output from each of the sensors. At least one aspect of the physiological profile is communicated to a processor configured to analyze the at least one aspect and generate a physiological assessment based on the analysis. If desired, actuators can also be attached to the garment and actuated via the hub. By way of example but not limitation, a haptic device may be used to provide feedback to the individual.

The flow 100 includes coupling a plurality of sensors (and actuators if desired) to a compression garment 110, wherein the compression garment has stretchable portions that stretch (e.g., in a single dimension). The garment can include an undergarment, a base layer garment, an outer garment, and so on. The garment can include a shirt such as a short-sleeve shirt, a long-sleeve shirt, or a sleeveless shirt. The garment can include an activity-specific or gender-specific garment such as a sports bra. The garment can include a profession-specific garment such as a vest, a tactical vest, and the like. The garment can be machine washable. The garment can include one or more panels, where a panel can include a stretchable panel, a compression panel, and so on. In embodiments, the garment is made of multiple panels. The panels can include a front panel, back panel, left panel, right panel, top panel, back panel, etc. In other embodiments, the garment can be made of a single panel. The single panel can define one or more other panels. In embodiments, the single panel can be cut of material that defines a front and a back of the garment and the garment can include only a single (e.g., side) seam. The single side seam can be used to fix the garment to the body of the wearer of the garment. In other embodiments, the garment includes a treated material for chem-bio and biohazard environments. If desired, particularly where the garment needs to tightly conform to the body of the wearer, and particularly where it may be difficult to don such a tightly-conforming garment, the garment may include a zipper so that the unzipped garment can be easily donned but the zipped garment will tightly conform to the body of the wearer.

As described herein, the single panel or the multiple panels can be made of stretchable materials. The stretchable materials can be used to ensure that sensors can be positioned appropriately and can remain in position. The stretchable materials can improve comfort while the garment is being worn. In embodiments, the multiple panels can provide stretch directionality. The directionality of the stretch can include left-right stretch, in-out stretch, up-down stretch, and so on. The directionality of the stretch can accomplish a variety of design objectives. In embodiments, the stretch directionality enables total garment compression. Garment compression can aid in reducing blood pooling in limbs under high-G conditions, can assist with recovery after periods of high exertion, etc. The stretch directionality can help keep sensors in position. In embodiments, the stretch directionality can reduce motion artifact. The amount of stretch, the direction of stretch, and so on, can be controlled using modifying appliques. In embodiments, modifying appliques can be included to apply local compression and reduce motion artifact. The shapes of the appliques can include a cylindrical shape, a conical shape, and so on. One or more appliques can be applied to one or more panels, to positions on the garment adjacent to one or more sensor locations, etc. In embodiments, the appliques limit stretch of the material. The stretch of the material can be limited so that a sensor does not shift from its appropriate position on the body of the wearer of the garment. In embodiments, the appliques can increase friction between the garment and body. An increase in friction between the garment and the body can help keep sensors in position. The increase in friction can reduce movement or “riding up” of the garment. In further embodiments, the appliques can limit garment motion artifact. It should be appreciated that, as used herein, the term applique is meant to include substantially any materials which are added to the garment so as to modify the normal attributes of the garment, e.g., to limit stretch, to increase friction, etc. Thus, an applique may comprise stitching added to the garment, additional layers of fabric added to the garment, materials printed onto the garment, materials laminated onto the garment, etc.

Returning to the sensors, a variety of sensors can be included in the plurality of sensors mounted to the garment. In embodiments, at least one of the plurality of sensors is a heart rate sensor 112. The heart rate sensor can be based on a variety of sensing techniques such as EKG/ECG, EMG, EEG, and so on. In embodiments, the heart rate sensor can include a plurality of electrodes coupled to the garment, with a plurality of serpentine conductors connecting the electrodes to a hub. The electrodes can be placed on the garment to detect cardiac signals at appropriate positions on the body. The serpentine conductors are discussed below. Further sensors can be included in the plurality of sensors. The further sensors can include one or more of a skin temperature sensor, a galvanic skin response sensor, a blood volume pulse/photoplethysmography sensor, an activity sensor, and so on. The temperature sensor can include a body temperature sensor. The body temperature sensor can be used to determine skin temperature, body core temperature, environmental temperature, and the like. The further sensors can also include communication sensors, energy harvesting sensors, and pressure sensors. The sensors can also include affective computing sensors. Some or all of the sensors can be permanently affixed to the garment. Other sensors can be connected to the garment. In embodiments, the other sensors can include an inertial measurement unit (IMU), where the IMU can include a gyroscope, a magnetometer, an accelerometer, and so on. The sensors can include an altimeter. The sensors that can be connected to the garment can be connected in different configurations, can be removed for washing the garment or when not needed, etc.

The flow 100 includes mounting associated on-board electronics (e.g., the PUC) 120 that reside in or on the garment. The associated on-board electronics can control the one or more sensors by providing appropriate signals such as signals based on a frequency, a number of pulses, etc. The associated on-board electronics can capture data from the one or more sensors. The associated on-board electronics can provide a power source to the sensors. In embodiments, the power source can include a battery. The battery can include a sealed lead acid (SLA) or NiMH or zinc-oxide battery, a lithium-ion battery, a lithium-iron-phosphate (LiFePO4) battery, etc. Other power sources can also be provided. In embodiments, the associated on-board electronics may include a power source like an energy harvesting component. The energy harvesting component can capture energy such as solar energy, motion energy from body motion, thermal energy from body heat, and the like. Connections can be formed between the mounted associated on-board electronics and sensors, between the mounted associated on-board electronics and actuators (such as a haptic feedback device), and so on. The connections can be accomplished using conductors, where the conductors can include serpentine conductors. The serpentine conductors can be applied to the garment, pressed onto the garment, woven into the garment, and so on. The serpentine conductors can include a variety of geometric shapes. In embodiments, the serpentine conductors can be disposed in a straight line, a horseshoe, a curve, a sine or zigzag line, and the like. The serpentine conductors connect the sensors to a hub. The associated on-board electronics also connect to the hub, so that the associated on-board electronics are electrically connected to the sensors. The mounting of the associated on-board electronics can be accomplished using a clip, strap, pouch, box, and so on, that can couple an electronic component to the garment. In other embodiments, the associated on-board electronics can be removably attachable to the hub such as with the physiological unit computer (PUC) construction.

Thus it will be seen that associated on-board electronics are connected to the hub to operate the sensors and collect data. In one form of the invention, the associated on-board electronics may be permanently connected to the hub. In another form of the invention, the associated on-board electronics may be temporarily connected to the hub, e.g., such as with the PUC configuration.

In embodiments, the garment can connect to one or more external devices. The external devices can include devices used by the wearer of the garment while she or he is exercising, working, engaging in firefighting, law enforcement, or military activities, and so on. In embodiments, the one or more external devices can include goggles. The goggles can be coupled to one or more screens, where the screen can provide information to the wearer. The goggles can include a variety of “reality” goggles, where the goggles can include virtual reality (VR) goggles, augmented reality (AR) goggles, mixed-reality goggles (MR), extended reality (XR) goggles, and so on. The one or more external devices can include input devices. The input devices can include a camera or imaging device, a microphone or audio capture device, and the like. In embodiments, an external device can enable machine vision based on mono, stereo, or laser input. The machine vision can be based on visible light, infrared or near-infrared light, and so on. In embodiments, the one or more external devices can include night vision (NV) goggles.

The flow 100 includes generating a physiological profile 130 with the associated on-board electronics based on at least one output from the sensors. A physiological profile can include a variety of physiological information related to an individual wearing the garment. The physiological profile can include respiratory rate, heart rate, heart rate variability, skin temperature, core temperature, and so on. The physiological profile can further include galvanic skin response, blood volume based on pulse or photoplethysmography, activity, and so on. The physiological profile generated for an individual can be compared to a baseline profile for the individual, an average baseline derived from multiple individuals, and the like. The physiological profile can be compared to an average profile. Aspects of the physiological profile can be compared to a threshold, where a threshold can indicate “safe”, “not safe”, etc. Generating the physiological profile can be accomplished using circuitry designed and configured to be temporarily attached (block 132) to the garment at a hub. The associated on-board electronics can be based on a component such as a PUC. In embodiments, the associated on-board electronics can be coupled to the compression garment to provide at least a portion of the generating of the physiological profile.

The flow 100 includes communicating at least one aspect (step 140) of the physiological profile to a processor configured to analyze the at least one aspect. The communicating can be based on wired techniques, wireless techniques, hybrid techniques, and so on. The wireless techniques, for example, can include Wi-Fi™, Bluetooth™, Zigbee™, near-field communication (NFC), infrared, and so on. The processor to which the at least one aspect of the physiological profile is communicated can include a computing device such as a laptop computer, a personal electronic device such as a smartphone, personal digital assistant (PDA), or tablet, a desktop computer, a server, and so on. In embodiments, the processor can be a processor operated by the wearer of the garment. The configuring of the processor can be accomplished using software such as computer code, a program, an app, a function, a routine, and so on. The software can be uploaded by a user, downloaded over a computer network such as the Internet, and the like.

The flow 100 includes generating a physiological assessment (step 150) based on the analysis. The physiological assessment can be based on an average, a goal, a threshold, a zone, and so on. The physiological assessment that is generated can be shared with or provided to the wearer of the garment, with a team leader, with researchers, with command, and the like. In embodiments, the physiological assessment can be provided in real-time to an individual wearing the garment through visible or audible feedback, haptic feedback, and so on. The visible means can include an indicator such as a flashing light, an icon, a text warning on a screen, and so on. The audible means can include a buzzer or beeper, a tone, a spoken message, and the like. The assessment can also be provided using a technique such as haptic feedback. In embodiments, the assessment can provide a warning to the individual based on an aberration in the physiological profile. The aberration can include an elevated heart rate, a low heart rate, aberrant respiratory rate, excessive core or skin temperature, and the like. In embodiments, the assessment can include a graphical representation of the at least one aspect of the physiological profile. The graphical representation can include a gauge, a number, a “thermometer” indicator based on safe, caution, and danger, etc. In embodiments, the assessment can include at least one indirect measurement derived from multiple sensor readings. Various indirect measurements can be made. In embodiments, the indirect measurement can include a derived heart rate, respiration rate, skin conductance level, or skin conductance response. The indirect measurement can be performed using optical techniques. In other embodiments, the indirect measurement can include an activity classification. The activity classification can include classifying an activity as constituting marching, running, swimming, falling, inactivity, and the like. In embodiments, the activity classification can include sitting, standing, lying down, or walking. Note that the quality of the assessment relies heavily on the quality of the data than can be collected from the one or more sensors. In embodiments, the assessment can include an estimation of data quality for at least one output from a first sensor, based on at least one output from a second sensor. The second sensor can include a sensor selected at random from the plurality of sensors, a reference sensor, etc. In embodiments, the assessment can include an estimation of an individual's activity and posture. This aspect of the assessment can be particularly critical when the wearer of the garment is engaged in dangerous or risky activities. In a usage example, a firefighter can be detected to be lying down and not moving. This situation could result from the firefighter assessing a fire situation or could indicate that the firefighter is unconscious and in urgent need of assistance. In further embodiments, the assessment can include an estimation of anomalous behavior. The anomalous behavior can result from the wearer of the garment experiencing an emergency situation, suffering an injury, etc.

Thus, with reference to FIG. 1, flow 100 comprises coupling sensors (and, if desired, actuators) to a garment, coupling conductors to the garment and to the sensors, and coupling hubs to the garment and to the conductors, so that data from the sensors is delivered to the hubs. Associated on-board electronics may be mounted to the garment and connected to the sensors via the hubs so as to produce a physiological profile. The physiological profile is then communicated to a processor, e.g., a laptop or a smartphone, for generation of an assessment of the wearer's physiological state.

The associated on-board electronics may be “built into” the hub (or hubs); or the associated on-board electronics may be in the form of a PUC which is removably mountable to the hub (or hubs); or the associated on-board electronics may be separately mounted to the garment (formed in or secured on) and then electrically connected to the sensors (e.g., via the hub or hubs).

Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors. Various embodiments of the flow 100 can be included in an apparatus.

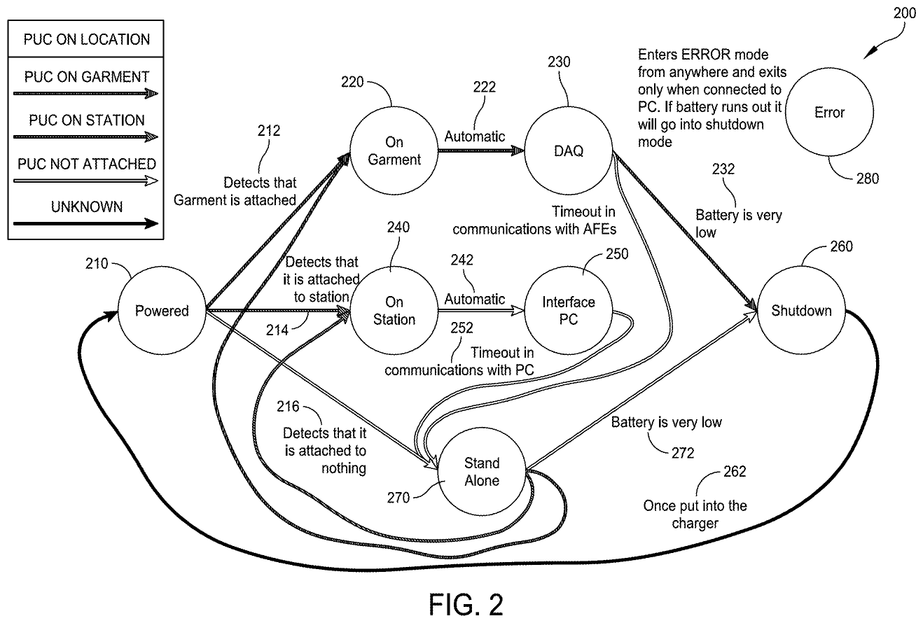

FIG. 2 illustrates a state diagram for PUC operation. The PUC generally functions as associated on-board electronics which are releasably mountable to the hubs and which operate one or more sensors coupled to a garment so as to provide the physiological profile. Operating the sensors can include providing a voltage, a current, a frequency, a pulse train, or other signal that can be required in order for the sensor to operate. The PUC can operate in any of a variety of states, where the state can be based on the location of the PUC. The various states can enable the PUC, where the PUC can enable physiological analysis using a wearable sensor array. A plurality of sensors and conductors are coupled to a compression garment, wherein the compression garment has stretchable portions that stretch in a single dimension, and wherein the conductors are connected to a hub. Associated on-board electronics are also connected to the hub. These associated on-board electronics (e.g., the PUC) enable a physiological profile to be generated. At least one aspect of the physiological profile is communicated to a processor configured to analyze the at least one aspect and generate an assessment based on the analysis. If desired, actuators can also be attached to the garment and actuated via the hub. By way of example but not limitation, a haptic device may be included as part of the wearable sensor array and used to provide feedback to the individual.

A state diagram for PUC operation is shown at 200. The states that can control the operation of the PUC can be based on one or more locations of the PUC. In embodiments, the one or more locations of the PUC can include the PUC coupled to a garment, the PUC in a station or charging dock, the PUC not attached to a garment, the PUC in an unknown state, and so on. Note that any state within the state diagram for the PUC technique can transition to the error state 280 (see below). The state diagram can enter the error state based on a fault, a failure, etc. associated with the operation of the PUC, a fault or failure associated with one or more sensors or actuators, a fault or failure associated with an external device, and so on. The PUC can remain in the error state until the PUC is connected to the “station” or charging dock. In the event that a power source powering the PUC is exhausted, the PUC can be placed in a shutdown mode or state 260 (see below).

The PUC “on garment” location can include a “powering on” state of the PUC (state 210). The powering on can be accomplished by using a power switch, connecting a power source to the PUC, and so on. The PUC can detect that it is attached to the garment (state 212) and can enter the on-garment state (state 220). The PUC can automatically transition to a data acquisition state (DAQ) (state 222). In the data acquisition state, data can be acquired from the plurality of sensors coupled to the garment. Data acquisition can continue while the PUC is attached to the garment, until the PUC is removed from the garment, or the power source is exhausted. If the battery charge, for example, becomes low (state 232), the PUC can transition to a shutdown state (state 260). The PUC can remain in the shutdown state until the PUC is placed into the charging dock (state 262). When sufficient power is available to the PUC, the PUC can return to the powered state (state 210). In another embodiment, the PUC can operate in a standalone mode. The PUC can begin in a standalone state (state 270). When the PUC is placed on garment, the PUC state can proceed to the on-garment state (state 220). The operating states through which the PUC operates can proceed as previously described for the PUC on garment location.

The PUC “on station” location or “charging dock” location can include powering on the PUC (state 210). The PUC can detect that it is attached to the station (state 214) and can proceed to an on-station state (state 240). Alternatively, the PUC can arrive at the on-station state from the standalone state (state 270). The PUC can automatically (state 242) transition to an interface with a PC (state 250). The interfacing with the PC (state 250) can include uploading collected sensor data, downloading configuration data or firmware, providing power for charging the PUC, and so on. The PUC can detect a timeout in communication with the PC (state 252), which can transition the PUC to a standalone state (state 270). The PUC “not attached location” can include powering on the PUC (state 210). The PUC can detect that it is not connected to a garment, is not attached to a station or loading dock, etc. (state 216). The PUC can proceed to the stand-alone state based on detecting the “not connected” condition. The stand-alone state can be entered from further states. In embodiments, the stand-alone state can be entered from the data acquisition state (state 230). The PUC can transition from the data acquisition state to the stand-alone state by disconnecting or detaching the PUC from the garment. The stand-alone state can further be entered from the “interface with PC” state 250. The PUC can transition from the interface PC state to the stand-alone state by removing the PUC from the charging dock or station. The PUC can transition from the stand alone state to the shutdown state 260 based on an exhausted power source or low battery condition (state 272). The PUC in an unknown location can include the PUC being in the shutdown state 260. The unknown “location” can include configuring the PUC for the first time, obtaining a PUC for deployment on a garment, and so on. The PUC can be placed in a charging dock (state 262) in order to charge a power source coupled to the PUC. When a sufficient level of charge has been obtained by the power source coupled to the PUC, the state of the PUC can proceed to the powered state 210.

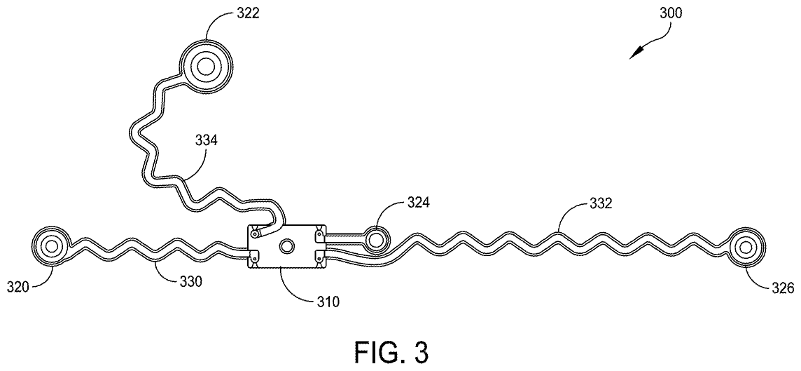

FIG. 3 shows sensors and serpentine conductors, where the serpentine conductors are shown in both straight and curved configurations. As discussed herein, sensors, actuators, and so on can be coupled to a compression garment or other garment. The garment can include a short-sleeve, long-sleeve, or sleeveless shirt, base layers, athletic or specialty clothing, outer clothing, and so on. The sensors and actuators can be mounted on the garment, woven into the garment, etc. The sensors and actuators can be coupled to a hub, where the coupling the sensors and actuators to the hub can be accomplished using conductors. The conductors, which can be printed on, woven into, mounted on, etc. the garment, can include various patterns that enable stretching of the garment based on expansion or compression of the body part on which the garment is worn. In embodiments, the conductor pattern is based on serpentine conductors, where the serpentine conductors can include a curved configuration. The serpentine conductors enable physiological analysis using a wearable sensor array. A plurality of sensors and actuators is coupled to a compression garment. Associated on-board electronics that reside in or on the garment are mounted to the garment and connected to the wearable sensor array. A physiological profile is generated by the associated on-board electronics based on at least one output from the sensors. At least one aspect of the physiological profile is communicated to a processor configured to analyze the at least one aspect and generate an assessment based on the analysis. In one preferred form of the invention, the associated on-board electronics communicate with the processor through wired or wireless means.

Sensors and serpentine conductors including straight and curved configurations are shown at 300. The serpentine conductors can couple one or more sensors to a hub 310. The hub can be used to attach, insert, apply, or otherwise couple a component to the garment. The component that can be coupled to the garment can include the associated on-board electronics in the form of a PUC (described below). The sensors, such as sensors 320, 322, 324, and 326, can be used to gather data from the wearer's body. The sensors can include sensors for measuring or detecting body temperature, galvanic skin response, blood volume pulse or photoplethysmography, activity, communications sensors, energy harvesting sensors, pressure sensors, affective sensors, etc. If desired, actuators can also be attached to the garment, connected to a hub via conductors, and actuated via the hub. By way of example but not limitation, a haptic device may be included as part of the wearable sensor array and used to provide feedback to the individual.

The sensors can be coupled to the hub using serpentine conductors arranged in a straight configuration such as serpentine conductors 330 and 332. Other serpentine conductors can include a curved configuration such as serpentine conductor 334. The curved serpentine conductor can be used to improve comfort for the person wearing the garment to which sensors are attached. The serpentine conductors can be printed onto the garment, woven into the garment, applied to the garment, and so on. The serpentine conductors can enable dimensional changes to the garment, such as stretching, while maintaining connectivity. The serpentine conductors can resist breaking by changing shape in compliance with the garment. The serpentine conductors can be based on a variety of shapes, patterns, geometries, etc. In embodiments, the one or more conductors can be in the shape of a straight line, a horseshoe, a sine or a “zigzag” pattern, a radial pattern, and the like.

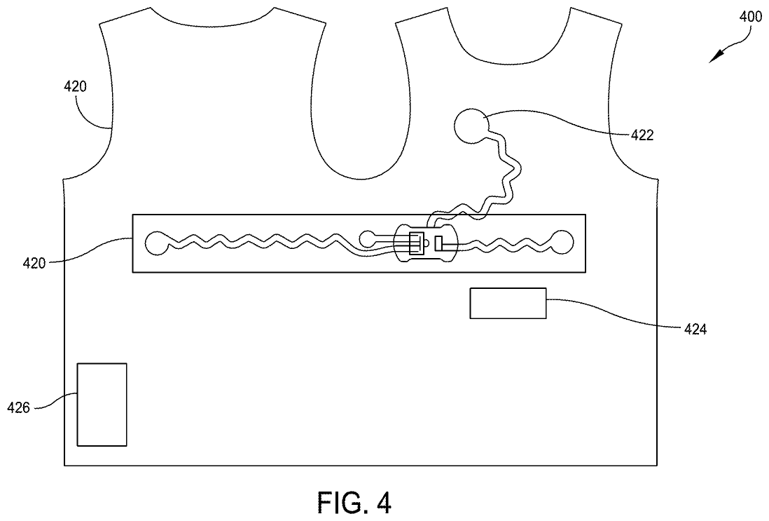

FIG. 4 illustrates a garment with sensors. A garment such as a compression garment can be worn by a person. The person can be a civilian, an athlete, a member of emergency services, law enforcement, or the military, and so on. Sensors can be coupled to the garment to determine a physiological profile for the person wearing the garment, and actuators can be coupled to the garment for providing haptic feedback to the person wearing the garment. As discussed herein, the garment can include a short-sleeve, long-sleeve, or sleeveless shirt, a base layer garment, an outer garment such as a vest, and so on. A plurality of sensors and actuators can be coupled to a compression garment, where the compression garment has stretchable portions that stretch, e.g., in a single dimension. Associated on-board electronics are mounted to the garment (e.g., they reside in or on the garment), and these electronics are configured to generate a physiological profile based on at least one output from the sensors. The associated on-board electronics can be mounted to the garment and connected to the hub by conductors; or the associated on-board electronics can be built into the hub; or the associated on-board electronics may be temporarily attached to the garment at a hub (e.g., the associated on-board electronics may be in the form of a PUC which releasably mounts to the hub). The associated on-board electronics are used to generate a physiological profile based on at least one output from the sensors. At least one aspect of the physiological profile is communicated to a processor, e.g., by wired or wireless means. The processor is configured to analyze the at least one aspect and generate a physiological assessment based on the analysis.

An illustration of a garment and sensors is shown at 400. The design of the garment can be based on anthropomorphic data collected from a population of wearers of such a garment, potential wearers of the garment, and so. The garment can be provided in a range of clothing sizes such as extra small, small, medium, large, extra-large, and so on. The garment can be sized differently for males or females. The garment can be designed to be worn on a human body by pulling the garment over the head of the wearer, by fastening one or more edges of the garment, and so on. The garment can comprise a single panel or multiple panels. In embodiments, the single panel is cut of material that defines a front and a back of the garment and the garment includes only a single (e.g., side) seam. The multiple panels can include a front or back panel, a left side or right side panel, a top or bottom panel, etc. In embodiments, the garment made from multiple panels of material can include a stretchable material, a material for a compression garment, and so on. The multiple panels can provide stretch in all directions, or the multiple panels can provide stretch with selected directionality. The directionality of the stretch can be modified by mounting appliques to the garment. The modifying appliques can be included to apply local compression and reduce motion artifact. The stretch directionality can include vertical stretch, horizontal stretch, and the like. In embodiments, the stretch directionality can enable total garment compression. The total garment compression can be used to improve physiological sensing, can improve recovery after strenuous exercise, etc.

Various sensors and actuators, conductors, and hubs can be mounted to the garment 410. In embodiments, the one or more sensors can include a respiration sensor band 420. The respiration sensor can measure chest expansions resulting from breathing, chest excursion based on the depth of the breathing such as shallow breathing, deep breathing, gasping, etc. Other sensors can be included. The one or more sensors can include a heart rate sensor 422. The heart rate sensor can detect a heartbeat, a heart rate, heart rate variability, and so on. Further sensors, such as sensor 424, can be coupled to the garment. In embodiments, the further sensors can include a temperature sensor, an orientation sensor, and the like. The respiration band, the heart rate sensor, and other sensors, or actuators, can be connected to a hub 426 using a conductor. In a preferred form of the invention, the sensors are connected to the hub 426 using serpentine conductors. The serpentine conductors can expand and contract based on breathing, body motion, and so on. A PUC, described herein, can be mounted to the hub. The PUC can generate a physiological profile for the wearer of the garment and can communicate an aspect of the physiological provide to a processor. Alternatively, the associated on-board electronics can be built into the hub or disposed elsewhere on the garment and electrically connected to the sensor array (e.g., via hub 426).

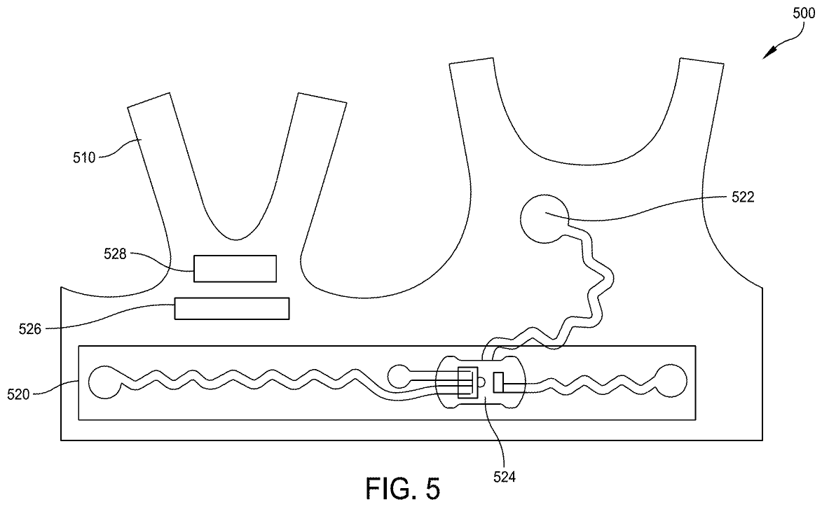

FIG. 5 shows a garment and sensors for females at 500. Sensors, actuators, conductors, hubs and so on can be coupled to a garment such as a compression garment. As discussed herein, the garment can include a short-sleeve, long-sleeve, or sleeveless shirt, a base layer garment, an outer garment such as a vest, and so on. In embodiments, the garment can be designed for use by females. The garment designed for use by females enables physiological analysis using a wearable sensor array. A plurality of sensors and actuators, conductors and hubs are coupled to a compression garment, where the compression garment has stretchable portions that stretch, e.g., in a single dimension. Associated on-board electronics are mounted (reside in or on) the garment, and these associated on-board electronics are configured to generate a physiological profile based on at least one output from the sensors. The associated on-board electronics can be mounted to the garment and electrically connected to the hub by conductors; or the associated on-board electronics can be built into the hub; or the associated on-board electronics may be temporarily attached to the garment at a hub (e.g., the associated on-board electronics may be in the form of a PUC which releasably mounts to the hub). The associated on-board electronics are used to generate a physiological profile based on at least one output from the sensors. At least one aspect of the physiological profile is communicated to a processor, e.g., by wired or wireless means. The processor is configured to analyze the at least one aspect and generate an assessment based on the analysis.

A garment 510 designed for use by females is shown in FIG. 5. The design of the garment can be based on anthropomorphic data collected from a population of wearers of such a garment. A garment can be designed to be applied to a human body by pulling the garment over the head of the wearer, by fastening one or more edges of the garment, and so on. The garment can comprise a single panel or multiple panels, such as a front or back panel, a left side or right side panel, a top or bottom panel, etc. In embodiments, the garment made from multiple panels of material can include a stretchable material, a material for a compression garment, and so on. The multiple panels can provide stretch in all directions, or the multiple panels can provide stretch with selected directionality. The stretch directionality can include vertical stretch, horizontal stretch, and the like. In embodiments, the stretch directionality can enable total garment compression. The total garment compression can be used to improve physiological sensing of the person wearing the garment, can improve recovery after strenuous exercise, etc. In embodiments, the single panel is cut of material that defines a front and a back of the garment and the garment includes only a single (e.g., side) seam.

Various sensors and actuators, conductors and hubs can be mounted to the garment 510. In embodiments, the one or more sensors can include a respiration sensor band 520. The respiration sensor can measure chest expansions resulting from breathing, chest excursion based on the depth of the breathing such as shallow breathing, deep breathing, gasping, etc. The one or more sensors can include a heart rate sensor 522. The heart rate sensor can detect a heartbeat, a heart rate, heart rate variability, and so on. The respiration band, the heart rate sensor, and so on, can be connected to a hub 524 using a conductor. In a preferred form of the invention, the sensors are connected to the hub using serpentine conductors. The serpentine conductors can expand and contract based on breathing, body motion, and so on. A PUC, described herein, can be mounted to the hub. The PUC can generate a physiological profile for the wearer of the garment and can communicate an aspect of the physiological provide to a processor, e.g., by wired or wireless means. Alternatively, the associated on-board electronics can be built into the hub or disposed elsewhere on the garment and electrically connected to the sensor array (e.g., via the hub 524). The processor is configured to analyze the at least one aspect and generate an assessment based on the analysis. Further sensors, such as sensor 526, sensor 528, and so on, can be coupled to the garment. In embodiments, the further sensors can include a temperature sensor, an orientation sensor, and the like.

In one preferred form of the invention, the sensors and actuators, conductors and hubs comprise an assembly which may sometimes be referred to herein as an array, or as a sensor array, or as a wearable sensor array. This sensor array may or may not incorporate the aforementioned associated on-board electronics therein, but in any case this sensor array is connected to the aforementioned associated on-board electronics.

It should be appreciated that the sensor array may be mounted to the garment so that the sensor array faces the skin of the wearer, which can be helpful where the sensors of the sensor array need to directly contact the skin of the wearer. Alternatively, the sensor array may be mounted to the garment so that the sensor array faces away from the skin of the wearer, which can be helpful where the sensors of the sensor array may need to be easily accessed while the garment is being worn. In this latter case, if the sensor array is mounted to the garment so that the sensor array faces away from the skin of the wearer, and if the sensors of the sensor array need to directly contact the skin of the wearer, the garment may include holes aligned with the sensors of the sensor array so as to allow the sensors to directly contact the skin of the wearer.

It should be appreciated that the various components of the sensor array may be individually mounted onto a garment, or the various components of the array may be separately assembled as a subsystem and then the subsystem mounted as a unit onto a garment.

It should also be appreciated that the various components of the array may be individually mounted onto a portion of a garment, and then that portion of the garment attached to the remainder of the garment; or the various components of the array may be separately assembled as a subsystem, the subsystem mounted as a unit onto a portion of a garment, and then that portion of the garment attached to the remainder of the garment.

It should also be appreciated that the associated on-board electronics (which are electrically connected to the sensor array and use the sensor data to generate a physiological profile which is then forwarded to a processor which generates a physiological assessment) may be mounted to the sensor array prior to the sensor array being mounted to the garment or after the sensor array has been mounted to the garment. By way of example but not limitation, the associated on-board electronics may be secured to the garment and then permanently connected (e.g., via soldering) to the sensor array at the hub; or the associated on-board electronics may be secured to the garment and then temporarily connected (e.g., via a releasable connector) to the hub; or the associated on-board electronics may be formed as part of the hub; or the associated on-board electronics may be secured to the hub after the hub has been mounted to the garment (e.g., such as with the PUC construction disclosed herein).

Furthermore, it should be appreciated that the associated on-board electronics may be provided in a modular scheme, i.e., a basic system may have one level of computing power, with the system having the provision of being able to add additional electronics modules so as to provide additional levels of computing power “on demand”. By way of example but not limitation, the hub might comprise a USB-type connector allowing one or more modules (e.g., PUCs) to be serially chained onto the hub, whereby to allow additional computing power to be added on demand.

Note that, if desired, heat mitigators may be disposed on the garment between the associated on-board electronics and the garment, or between the garment and the skin of the wearer, so as to protect the wearer from heat generated by the associated on-board electronics.

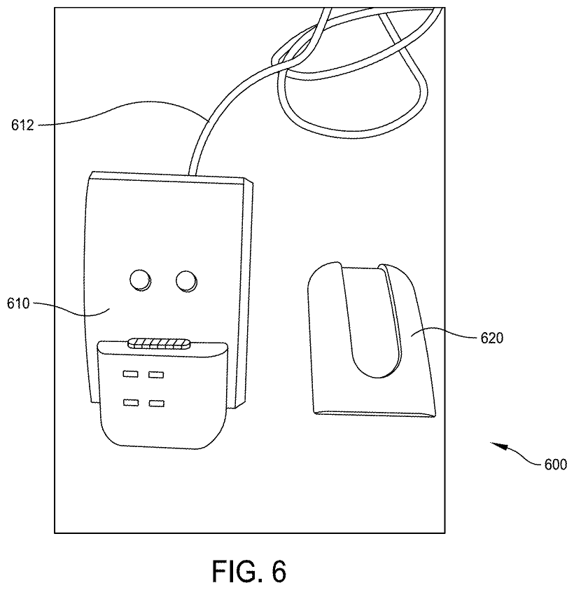

FIG. 6 shows a PUC and charging dock. As described herein, the PUC can be mounted on a garment (e.g., by plugging into the hub) and can collect data from one or more sensors coupled to the garment. The PUC can include a power source such as a rechargeable battery, an energy harvesting source, and so on. The power source coupled to the PUC can require charging. Charging the PUC enables physiological analysis using a wearable sensor array. Sensors and actuators, conductors and hubs are coupled to a compression garment. The compression garment has stretchable portions that stretch in a single dimension. Associated on-board electronics are connected to the hub. These associated on-board electronics enable a physiological profile to be generated, based on at least one output from each of the sensors. As noted above, these associated on-board electronics can be connected to the garment and electrically connected to the hub; or the associated on-board electronics can be built into the hub; or the associated on-board electronics can be temporarily attached to the garment at the hub; or the associated on-board electronics may be in the form of a PUC which releasably mounts to the hub. At least one aspect of the physiological profile is communicated from the associated on-board electronics to a processor configured to analyze the at least one aspect of the physiological profile and to generate an assessment based on the analysis.

A PUC and charging dock are shown at 600. A charger 610 can include a desktop charger. While a desktop charger is shown, the charger can include a portable charger, a travel charger, a field charger, and so on. The charger such as the desktop charger can include a tether or umbilical 612. The umbilical can be used to couple the charger to a computing device such as a laptop computer, a desktop computer, a computer that has met military standards, and the like. The umbilical can be used to provide power to the charging dock, to communicate with the dock, to transfer data between the PUC and the computing device, etc. A PUC 620 is shown adjacent to the charging dock. The PUC is coupled to the charging dock to charge the PUC, to transfer data to or from the PUC, and so on.

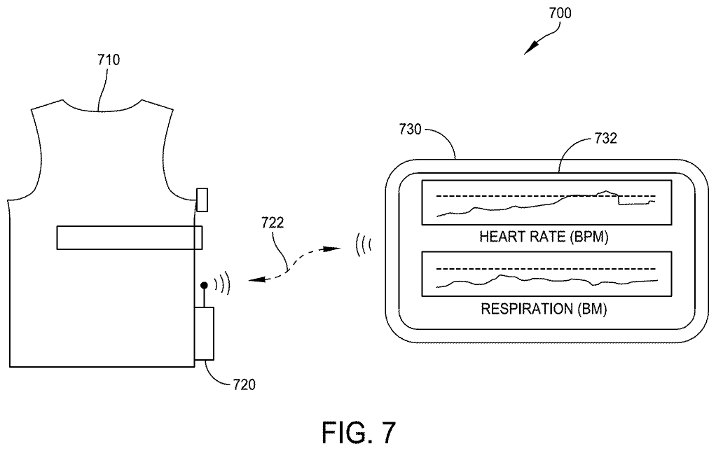

FIG. 7 is a block diagram showing data processing in the system. This system comprises the associated on-board electronics (e.g., a PUC) which collect data from the sensors and provides the physiological profile. Physiological data associated with a person can be collected using a variety of sensors. The sensors can include respiration, heart rate, temperature, orientation, and other sensors. Data from the sensors can be collected and analyzed. The collecting and the analyzing can be accomplished using the associated on-board electronics (e.g., a PUC). The associated on-board electronics (e.g., the PUC) enables at least one aspect of a physiological profile to be generated using a wearable sensor array. A plurality of sensors and actuators is coupled to a compression garment. The compression garment has stretchable portions that stretch, e.g., in a single dimension. Associated on-board electronics are connected to the hub. These associated on-board electronics enable a physiological profile to be generated, based on at least one output from the sensors. At least one aspect of the physiological profile is communicated from the associated on-board electronics to a processor which is configured to analyze the at least one aspect and generate an assessment based on the analysis.

A block diagram for a PUC-based system is shown at 700. The PUC can be mounted to a hub on a garment in order to power one or more sensors, collect sensor data, communicate sensor data, and so on. The one or more sensors can be attached to a garment 710, embedded in the garment, and so on. The garment can include a garment worn on the upper body of a human, the lower body, and so on. The garment can include a short-sleeve shirt, a long-sleeve shirt, a sleeveless shirt, and so on. The garment can include a base layer garment. The garment can include an activity-related garment such as a sports bra. The garment can include an outer layer garment such as a vest, coat, brush-proof or waterproof trousers, and so on. A PUC 720 can be mounted to the garment at a hub. The hub can include a clip, a strap, a pouch or pocket, a bracket, and so on. The hub can include access to serpentine conductors, where the serpentine conductors can be coupled to one or more sensors associated with the garment. The PUC can enable communicating (see 722) an aspect of a physiological profile to a processor, where the processor can be configured to analyze the aspect of the physiological profile. The communicating can be accomplished using a variety of wired or wireless communication techniques. In embodiments, the communicating can be accomplished using one or more wireless techniques such as Wi-Fi™, Bluetooth™, Zigbee™, infrared (IR), near-field communication (NFC), etc.

The processor that can receive the aspect of the physiological profile communicated by the PUC can include a computing device such as a laptop computer, a personal electronic device such as a smartphone, a personal digital assistant (PDA), a tablet, and the like. In the figure, the PUC 720 can communicate the aspect of the physiological profile to a tablet 730. The tablet can process the aspect of the physiological profile using code, an app, and so on. The tablet can render results of the physiological analysis on a display 732 associated with the tablet. The analysis results that can be rendered on the display can include a heart rate over time in units such as beats per minute. The analysis results can include a respiration rate in units such as breaths per minute. Further data communicated by the PUC can be analyzed on the electronic device 730. The further data can include a temperature, a body orientation, blood pressure, blood volume pulse, etc.

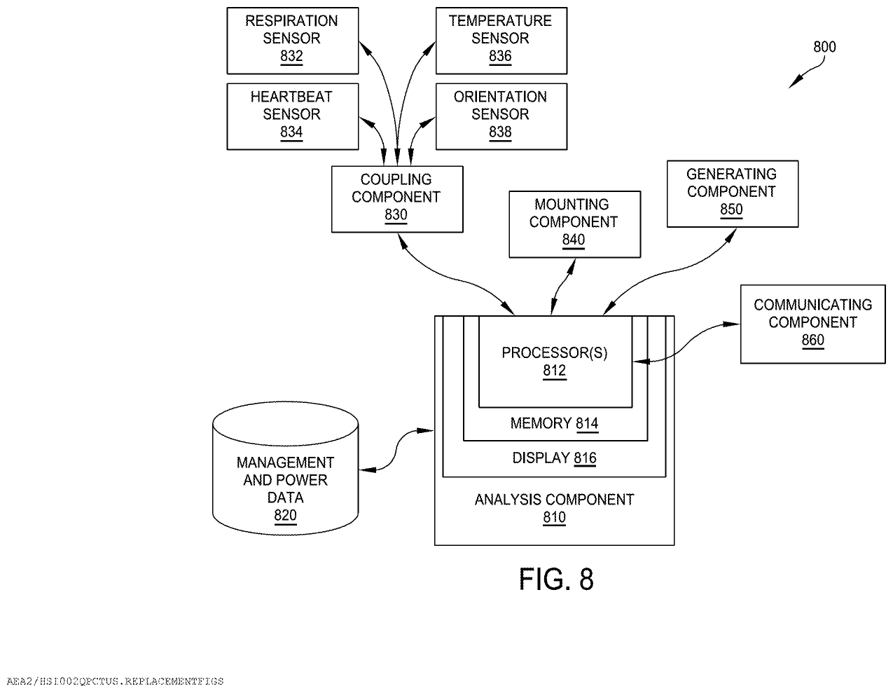

FIG. 8 is a system diagram for physiological analysis using a wearable sensor array. The sensor array can be coupled to a garment such as a shirt, a base layer, a sports bra, a vest, a compression garment, and so on. A plurality of sensors and actuators, conductors and hubs are coupled to a compression garment, where the compression garment has stretchable portions that stretch in a single dimension. Associated on-board electronics are connected to the hub. These associated on-board electronics enable a physiological profile to be generated, based on at least one output from each of the sensors. At least one aspect of the physiological profile is communicated to a processor which is configured to analyze the at least one aspect and generate an assessment based on the analysis.

The system 800 can include an analysis component 810. The analysis component can include one or more electronic components which can be used to monitor and control sensors within the wearable sensor array. The analysis component 810 can comprise one or more processors 812, a memory 814 coupled to the one or more processors 812, and a display 816. The display 816 can be configured and disposed to present collected data, analysis, physiological profiles, intermediate analysis steps, instructions, algorithms, or heuristics, a thermal signature, heating data, and so on.

The system 800 can include a management and power data component 820. The management data can include a library of lookup tables, physiological sensor characteristics, functions, algorithms, routines, code segments, apps, and so on, that can be used for management of the one or more physiological sensors. The power data can include status of a source of electrical power, power dissipation data for the one or more sensors, power dissipation for an analysis component, and so on. The system 800 can include a coupling component 830. The coupling component can act as an interface between one or more sensors and the analysis component 810. The coupling component can further act as an interface between the one or more sensors and a garment that can be worn on a human body. The coupling component can provide power or operating signals to the one or more sensors, can capture sensor data, etc. The coupling component can act as an interface between the one or more sensors and a wearable garment to which the one or more physiological sensors can be coupled. The coupling can include enabling or disabling one or more the wearable physiological sensors, monitoring wearable power system status data such as voltage, current, or temperature, and the like. In embodiments, the one or more physiological sensors can include a respiration sensor 832. The respiration sensor can be used to detect breathing, a breathing rate, a breathing amount such as shallow breathing or deep breathing, and so on. In embodiments, the one or more physiological sensors can include a heart rate sensor 834. The heart rate sensor can detect a heartbeat, a heart rate, heart rate variability, and the like. In other embodiments, the one or more physiological sensors can include a temperature sensor 836. The temperature sensor can detect skin temperature, and infer an internal temperature, can measure an internal temperature with a probe, etc. In further embodiments, the one or more physiological sensors can include an orientation sensor 838. The orientation sensor can be used to determine wither the wearer of the sensor is upright, prone, and so on.

The system 800 can include a mounting component 840. The mounting component can enable mounting associated on-board electronics that are associated with a garment. The mounting component can enable mounting the associated on-board electronics that reside in or on the garment. In embodiments, the mounting component can include a hub, where the hub can be associated with the garment. The hub can be connected to one or more electrodes, sensors, and so on, using one or more serpentine conductors. The system 800 can include a generating component 850. The generating component can generate a physiological profile based on at least one output from each of the sensors discussed previously. The generating can be accomplished using circuitry designed and configured to be temporarily attached to the garment at a hub. The hub can be coupled to the garment. The hub can enable mounting of a processor, an analyzer, and the like. The system 800 can include a communicating component 860. The communicating component can communicate at least one aspect of the physiological profile to a processor configured to analyze the at least one aspect and generate an assessment based on the analysis. The communicating component can enable wired communication, wireless communication, and so on.

In one preferred form of the invention, coupling component 830 and mounting component 840 may comprise the aforementioned hubs, and generating component 850 and various others of the components shown in FIG. 8 may comprise the aforementioned associated on-board electronics (e.g., a PUC).

Depending on the nature of the sensors acquiring the physiological data, various processing techniques may be utilized to optimize the usability of the physiological data.

By way of example but not limitation, where the physiological data relates to electrocardiogram/heart rate (ECG/HR), the following processing techniques may be utilized:
Processing Lead (i.e., Channel) Selection: the lead selected for HR calculation may be based on which lead results in minimized noise;
Signal Quality Calculation:
   the measurement of noise in the isoelectric region of ECG signal may be combined with the measurement of R-peak prominence;
   this method can be used to distinguish between different levels of signal quality, e.g., the signal quality is “very good”, “acceptable” and “bad”—this allows the calculation of signal quality to be put into context, e.g., if the user wants to use the shirt for arrythmia detection, the user's definition of good signal quality may differ from a user who only needs to know their heart rate during exercise;
   use of a signal quality index (SQI) for lead selection;
   use of the SQI to improve/validate other derived parameters such as core temperature;
   use of SQIs (not only ECG, but also respiratory and any other physiological measurement modality that may be included on garments, such as photoplethysmography (PPG), electromyography (EMG), galvanic skin response (GSR), etc.) for an “automatic fit recommendation system”, i.e., the software/algorithm automatically tests the suitability of a garment/measurement system for an individual based on signal quality and makes recommendations about better fit.

By way of further example but not limitation, where the physiological data relates to respiration, the following processing techniques may be utilized:
A respiration signal quality index (SQI) may be calculated based on a comparison of two different rate calculation methods—if the difference is too large, then SQI (or confidence) is low;
Respiration rate calculation can be made more robust by combining results from multiple respiration rate extraction modalities, such as electrocardiogram (ECG) and photoplethysmography (PPG);
Classification of activities from respiration, such as yawning, talking, coughing, at least during rest, and the classification may be combined with inertial measurement unit (IMU) data.

By way of further example but not limitation, where the physiological data relates to activity, the following processing techniques may be utilized:
activity classification;
the extraction and combination of features and implementation of classification logic based on a single accelerometer disposed on the chest (rather than multiple accelerometers), optionally combined with multiple machine learning algorithms with an empirical threshold detection method for increased robustness.

By way of further example but not limitation, where the physiological data relates to posture detection, the following processing techniques may be utilized:
posture detection using Intexar, embroidered inductive measurement, or other similar sensors (which could at the same time be decorative or supporting elements) in strategic locations on the garment.

The system 800 can include a computer program product embodied in a non-transitory computer readable medium for physiological analysis, the computer program product comprising code which causes one or more central processing units (CPUs) to drive the associated on-board electronics which produce the physiological profile or to drive the processor which generates a physiological assessment based on the physiological profile.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”— may be implemented by computer program instructions, by special-purpose hardware-based computer systems, by combinations of special purpose hardware and computer instructions, by combinations of general purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A method for physiological analysis, the method comprising:
mounting a plurality of sensors, a plurality of conductors, and a hub to a compression garment, wherein the plurality of conductors electrically connect the plurality of sensors to the hub, and mounting associated on-board electronics to at least one of the compression garment and the hub and electrically connecting the associated on-board electronics to the hub;
using at least one sensor to acquire physiological data from a wearer of the garment;
using the associated on-board electronics to generate a physiological profile based on at least one output from the plurality of sensors;
communicating at least one aspect of the physiological profile to a processor located off garment; and
using the processor to analyze the at least one aspect of the physiological profile and generate a physiological assessment based on the analysis.

2. A method according to claim 1 wherein the compression garment has stretchable portions.

3. A method according to claim 2 wherein the stretchable portions stretch in a single dimension.

4. A method according to claim 1 wherein the sensors, conductors and hub comprise a sensor array.

5. A method according to claim 4 wherein the sensor array is mounted to the garment so that the sensor array faces the skin of the wearer.

6. A method according to claim 4 wherein the sensor array is mounted to the garment so that the sensor array faces away from the skin of the wearer.

7. A method according to claim 6 wherein the garment comprises holes aligned with the sensors of the sensor array so as to allow the sensors to directly contact the skin of the wearer.

8. A method according to claim 4 wherein the sensors, conductors and hub of the sensor array are individually mounted to the garment.

9. A method according to claim 4 wherein the sensors, conductors and hub of the sensor array are separately assembled as a subsystem, and then the subsystem mounted as a unit onto the garment.

10. A method according to claim 4 wherein the sensors, conductors and hub of the sensor array are individually mounted to a portion of the garment, and then that portion of the garment is attached to the remainder of the garment.

11. A method according to claim 4 wherein the sensors, conductors and hub of the sensor array are separately assembled as a subsystem, the subsystem mounted as a unit onto a portion of the garment, and then that portion of the garment attached to the remainder of the garment.

12. A method according to claim 1 wherein the associated on-board electronics are secured to the garment and then permanently connected to the hub.

13. A method according to claim 1 wherein the associated on-board electronics are secured to the garment and then temporarily connected to the hub.

14. A method according to claim 1 wherein the associated on-board electronics are formed as part of the hub.

15. A method according to claim 1 wherein the associated on-board electronics are secured to the hub after the hub has been mounted to the garment.

16. A method according to claim 1 wherein the associated on-board electronics are provided in one or more modules so as to enable the computing power of the associated on-board electronics to be adjusted “on demand”.

17. A method according to claim 16 wherein the hub comprises a USB-type connector allowing modules to be serially chained onto the hub.

18. A method according to claim 1 wherein a heat mitigator is disposed on the garment between the associated on-board electronics and one from the group consisting of the garment and the skin of the wearer.

19. A method according to claim 1 wherein the plurality of sensors comprise at least one from the group consisting of a heart rate sensor, a respiration sensor, a body temperature sensor, and an orientation sensor.

20. A method according to claim 1 wherein the plurality of conductors comprise at least one serpentine conductor.

21. A method according to claim 1 further comprising:
mounting at least one haptic device to a compression garment, wherein the at least one haptic device is electrically connected to the hub and further wherein the at least one haptic device is configured to selectively apply a physical signal to a wearer; and
using the physiological assessment provided by the processor to cause the associated on-board electronics to actuate the at least one haptic device so as to deliver a physical signal to the wearer.

22. A method according to claim 1 wherein the garment is made of multiple panels.

23. A method according to claim 22 wherein the multiple panels are made of stretchable materials.

24. A method according claim 22 wherein the multiple panels provide stretch directionality.

25. A method according to claim 24 wherein the stretch directionality enables total garment compression.

26. A method according to claim 24 wherein the stretch directionality reduces motion artifact.

27. A method according to claim 1 wherein the garment is made of a single panel.

28. A method according to claim 27 wherein the single panel is cut of material that defines a front and a back of the garment and the garment includes only a single seam.

29. A method according to claim 1 wherein modifying appliques are included to apply local compression and reduce motion artifact.

30. A method according to claim 29 wherein the appliques limit stretch of the material.

31. A method according to claim 29 wherein the appliques increase friction between the garment and body.

32. A method according to claim 29 wherein the appliques limit garment motion artifact.

33. A method according to claim 1 wherein the associated on-board electronics include a rechargeable battery power source.

34. A method according to claim 1 wherein the associated on-board electronics include an energy harvesting component power source.

35. A method according to claim 1 wherein the associated on-board electronics interact with a removable central processing unit.

36. A method according to claim 1 wherein the physiological assessment is provided in real-time to an individual wearing the garment.

37. A method according to claim 36 wherein the physiological assessment is provided in real-time through visible or audible feedback.

38. A method according to claim 36 wherein the physiological assessment provides a warning to the individual based on an aberration in the physiological profile.

39. A method according to claim 1 wherein the physiological assessment includes a graphical representation of the at least one aspect of the physiological profile.

40. A method according to claim 1 wherein the physiological assessment includes at least one indirect measurement derived from multiple sensor readings.

41. A method according to claim 40 wherein the indirect measurement includes a derived heart rate, heart rate variability, respiration rate, respiration volume, skin temperature, body core temperature, body hydration, skin conductance level, or skin conductance response.

42. A method according to claim 40 wherein the indirect measurement includes an activity classification.

43. A method according to claim 42 wherein the activity classification includes sitting, standing, lying down, or walking.

44. A method according to claim 1 wherein the physiological assessment includes an estimation of data quality for at least one output from a first sensor, based on at least one output from a second sensor.

45. A method according to claim 1 wherein the physiological assessment includes an estimation of an individual's activity and posture.

46. A method according to claim 1 wherein the physiological assessment includes an estimation of anomalous behavior.

47. A method according to claim 1 wherein the garment is machine washable.

48. A method according to claim 1 wherein the garment connects to external devices.

49. A system for physiological analysis comprising:
a compression garment;
a plurality of sensors mounted to the compression garment;
a plurality of conductors mounted to the compression garment;
a hub mounted to the compression garment, wherein the plurality of conductors electrically connect the plurality of sensors to the hub;
associated on-board electronics mounted to at least one of the compression garment and the hub, wherein the associated on-board electronics are electrically connected to the hub; and
a processor located off garment;
wherein at least one of the plurality of sensors acquires physiological data from a wearer of the compression garment;
wherein the associated on-board electronics generate a physiological profile based on at least one output from the plurality of sensors; and
wherein the processor analyzes the at least one aspect of the physiological profile and generates a physiological assessment based on the analysis.

50. A system according to claim 49 wherein the compression garment comprises stretchable portions.

51. A system according to claim 50 wherein the stretchable portions stretch in a single dimension.

52. A system according to claim 49 wherein the sensors, conductors and hub comprise a sensor array.

53. A system according to claim 52 wherein the sensor array is mounted to the garment so that the sensor array faces the skin of the wearer.

54. A system according to claim 52 wherein the sensor array is mounted to the garment so that the sensor array faces away from the skin of the wearer.

55. A system according to claim 54 wherein the garment comprises holes aligned with the sensors of the sensor array so as to allow the sensors to directly contact the skin of the wearer.

56. A system according to claim 52 wherein the sensors, conductors and hub of the sensor array are individually mounted to the garment.

57. A system according to claim 52 wherein the sensors, conductors and hub of the sensor array are separately assembled as a subsystem, and then the subsystem mounted as a unit onto the garment.

58. A system according to claim 52 wherein the sensors, conductors and hub of the sensor array are individually mounted to a portion of the garment, and then that portion of the garment is attached to the remainder of the garment.

59. A system according to claim 52 wherein the sensors, conductors and hub of the sensor array are separately assembled as a subsystem, the subsystem mounted as a unit onto a portion of the garment, and then that portion of the garment attached to the remainder of the garment.

60. A system according to claim 49 wherein the associated on-board electronics are secured to the garment and then permanently connected to the hub.

61. A system according to claim 49 wherein the associated on-board electronics are secured to the garment and then temporarily connected to the hub.

62. A system according to claim 49 wherein the associated on-board electronics are formed as part of the hub.

63. A system according to claim 49 wherein the associated on-board electronics are secured to the hub after the hub has been mounted to the garment.

64. A system according to claim 49 wherein the associated on-board electronics are provided in one or more modules so as to enable the computing power of the associated on-board electronics to be adjusted “on demand”.

65. A system according to claim 64 wherein the hub comprises a USB-type connector allowing modules to be serially chained onto the hub.

66. A system according to claim 49 wherein a heat mitigator is disposed on the garment between the associated on-board electronics and one from the group consisting of the garment and the skin of the wearer.

67. A system according to claim 49 wherein the plurality of sensors comprise at least one from the group consisting of a heart rate sensor, a respiration sensor, a body temperature sensor, and an orientation sensor.

68. A system according to claim 49 wherein the plurality of conductors comprise at least one serpentine conductor.

69. A system according to claim 49 further comprising:
mounting at least one haptic device to a compression garment, wherein the at least one haptic device is electrically connected to the hub and further wherein the at least one haptic device is configured to selectively apply a physical signal to a wearer; and
using the physiological assessment provided by the processor to cause the associated on-board electronics to actuate the at least one haptic device so as to deliver a physical signal to the wearer.

70. A system according to claim 49 wherein the garment is made of multiple panels.

71. A system according to claim 70 wherein the multiple panels are made of stretchable materials.

72. A system according to claim 70 wherein the multiple panels provide stretch directionality.

73. A system according to claim 72 wherein the stretch directionality enables total garment compression.

74. A system according to claim 72 wherein the stretch directionality reduces motion artifact.

75. A system according to claim 49 wherein the garment is made of a single panel.

76. A system according to claim 75 wherein the single panel is cut of material that defines a front and a back of the garment and the garment includes only a single seam.

77. A system according to claim 49 wherein modifying appliques are included to apply local compression and reduce motion artifact.

78. A system according to claim 77 wherein the appliques limit stretch of the material.

79. A system according to claim 77 wherein the appliques increase friction between the garment and body.

80. A system according to claim 77 wherein the appliques limit garment motion artifact.

81. A system according to claim 49 wherein the associated on-board electronics include a rechargeable battery power source.

82. A system according to claim 49 wherein the associated on-board electronics include an energy harvesting component power source.

83. A system according to claim 49 wherein the associated on-board electronics interact with a removable central processing unit.

84. A system according to claim 49 wherein the physiological assessment is provided in real-time to an individual wearing the garment.

85. A system according to claim 84 wherein the physiological assessment is provided in real-time through visible or audible feedback.

86. A system according to claim 84 wherein the physiological assessment provides a warning to the individual based on an aberration in the physiological profile.

87. A system according to claim 49 wherein the physiological assessment includes a graphical representation of the at least one aspect of the physiological profile.

88. A system according to claim 49 wherein the physiological assessment includes at least one indirect measurement derived from multiple sensor readings.

89. A system according to claim 88 wherein the indirect measurement includes a derived heart rate, heart rate variability, respiration rate, respiration volume, skin temperature, body core temperature, body hydration, skin conductance level, or skin conductance response.

90. A system according to claim 88 wherein the indirect measurement includes an activity classification.

91. A system according to claim 90 wherein the activity classification includes sitting, standing, lying down, or walking.

92. A system according to claim 49 wherein the physiological assessment includes an estimation of data quality for at least one output from a first sensor, based on at least one output from a second sensor.

93. A system according to claim 49 wherein the physiological assessment includes an estimation of an individual's activity and posture.

94. A system according to claim 49 wherein the physiological assessment includes an estimation of anomalous behavior.

95. A system according to claim 49 wherein the garment is machine washable.

96. A system according to claim 49 wherein the garment connects to external devices.

97. A method according to claim 2 wherein the stretchable portions stretch in more than a single dimension.

98. A method according to claim 1 wherein additional battery power is provided in one or more modules so as to extend operating time.

99. A system according to claim 50 wherein the stretchable portions stretch in more than a single dimension.

100. A system according to claim 49 wherein additional battery power is provided in one or more modules so as to extend the operating time of the system.
